(19) United States
(12) Patent Application Publication
(10) Pub. No.: US 2017/0248565 A1
(43) Pub. Date: Aug. 31, 2017

(54) MOLECULAR DETECTION APPARATUS AND MOLECULAR DETECTION METHOD

(71) Applicant: KABUSHIKI KAISHA TOSHIBA, Minato-ku (JP)

(72) Inventors: Ko YAMADA, Yokohama (JP); Hirohisa MIYAMOTO, Kamakura (JP); Reiko YOSHIMURA, Kawasaki (JP); Norikazu OSADA, Meguro (JP); Mitsuhiro OKI, Kawasaki (JP); Hiroko NAKAMURA, Yokohama (JP)

(73) Assignee: KABUSHIKI KAISHA TOSHIBA, Minato-ku (JP)

(21) Appl. No.: 15/258,447

(22) Filed: Sep. 7, 2016

(30) Foreign Application Priority Data
Feb. 29, 2016 (JP) 2016-037255

(51) Int. Cl. G01N 33/00

(52) U.S. Cl. CPC G01N 33/0013; G01N 33/0049

(57) ABSTRACT

A molecular detection apparatus according to an embodiment includes: a collection unit collecting a detection target gas containing a molecule to be detected; a substitution unit substituting a part of a molecular structure of at least a part of the molecule to generate a substitution product; a detector including a plurality of detection cells each having a sensor unit and an organic probe disposed at the sensor unit, the organic probe capturing the molecule or the substitution product; and a discriminator discriminating the molecule by a signal pattern based on an intensity difference of detection signals generated with the molecule or the substitution product being captured by the organic probes of the plurality of detection cells.

### CROSS REFERENCE TO RELATED APPLICATION

This application is based upon and claims the benefit of priority from Japanese Patent Application No. 2016-037255, filed on Feb. 29, 2016; the entire contents of which are incorporated herein by reference.

### FIELD

Embodiments described herein generally relate to a molecular detection apparatus and molecular detection method.

### BACKGROUND

A water heater or the like for household use is provided with a certain equipment that detects carbon monoxide generated when incomplete combustion occurs and is capable of notifying the risk thereof at an early stage. This kind of gas component considerably affects a human body. In this regard, according to the guidelines from LP gas safety committee, it is assumed that a carbon monoxide concentration of approximately 200 ppm (parts per million) causes the headaches. Various methods have been known for detecting the gas component having a relatively higher concentration. In contrast, however, the detection methods are limited for detecting the gas component having a concentration of ppb (parts per billion) to even ppt (parts per trillion), which corresponds to an extremely low concentration.

At a disaster site or a site at which an act of terrorism occurs or the like, it has been desired to sense the risk in advance by detecting the extremely small amount of the gas component. In many cases, such gas component having the extremely low concentration is detected by use of a large equipment in research facilities. In this case, a large sized installation type equipment, which is expensive and has large weight and volume, is required such as a gas chromatography or a mass spectrometer or the like. Under such circumstances, it has been demanded to provide an apparatus that is capable of detecting the gas component having an extremely low concentration on a real-time basis, in other words, an apparatus that has a smaller weight and volume and a better portability and is capable of detecting the gas component having the extremely low concentration in the order of ppt to ppb in a selective manner with higher sensitivity.

As a detection element for the gas component with the low concentration, for example, a certain element has been known that has a conductive layer in which a surface of a carbon nanostructure is surface modified with an organic substance or the like capable of selectively reacting or adsorbing with a specific substance and measures a potential difference or the like which varies depending on the gas component adhered to the surface of the carbon nanostructure. In this type of detection element, for example, when a similar component or the like to the detection target gas component is immixed as an impurity in a gas obtained in air, it is likely to fail to accurately detect the detection target gas component. Furthermore, in some cases, the organic substance which functions as a detection probe fails to sufficiently interact with a specific gas component. In this case, such gas component may fail to be detected with higher sensitivity. Accordingly, it is demanded to provide an apparatus that is capable of detecting the gas component having the extremely low concentration in a selective manner with higher sensitivity irrespective of the type of the gas component.

### BRIEF DESCRIPTION OF THE DRAWINGS

FIG. 1 is a block diagram illustrating a molecular detection apparatus according to a first embodiment.

FIG. 2 is a view illustrating a modification example to the molecular detection apparatus shown in FIG. 1.

FIG. 3 is a view illustrating a configuration of a detector of the molecular detection apparatus according to the first embodiment.

FIG. 4 is a view illustrating an example of a plurality of detection cells of the molecular detection apparatus according to the embodiment.

FIG. 5 is a view illustrating an example of a detection result of the to-be-detected molecule by the plurality of detection cells shown in FIG. 4.

FIG. 6 is a view illustrating an example of organic compounds used to an organic probe of a detector of the molecular detection apparatus according to the first embodiment.

FIG. 7 is a view illustrating an example of to-be-detected molecule detected by the molecular detection apparatus according to the first embodiment.

FIG. 8 is a view illustrating an example of substitution of the to-be-detected molecule in the molecular detection apparatus according to the first embodiment.

FIG. 9 is a block diagram illustrating a molecular detection apparatus according to a second embodiment.

FIG. 10 is a view illustrating an example of substitution of the to-be-detected molecule according to examples.

FIG. 11 is a view illustrating an example of a detected waveform of the to-be-detected molecule by the molecular detection apparatus according to the examples.

### DETAILED DESCRIPTION

According to an embodiment, a molecular detection apparatus is provided. The molecular detection apparatus includes: a collection unit collecting a detection target gas containing molecules to be detected; a substitution unit substituting a part of a molecular structure of at least a part of the molecules collected by the collection unit to generate a substitution product; a detector including a plurality of detection cells each including a sensor unit and an organic probe disposed at the sensor unit, the organic probe capturing the molecules or the substitution product; and a discriminator discriminating the molecules by a signal pattern based on an intensity difference of detection signals generated with the molecules or the substitution product being captured by the organic probes of the plurality of detection cells.

Hereinafter, a molecular detection apparatus and molecular detection method according to embodiments will now be described with reference to the accompanying drawings. In the embodiments, like or same reference numerals designate corresponding or identical configurations, and therefore such configurations may not be described repetitively. The drawings are schematically illustrated. For example, the relationship between a thickness and plane dimensions, a ratio of thicknesses of respective units and the like may differ from actual dimensions.

### First Embodiment

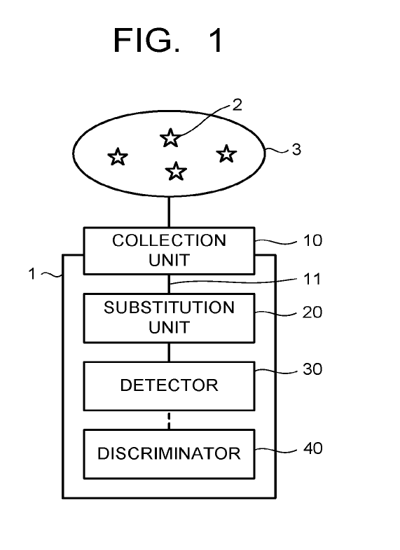

FIG. 1 is a block diagram illustrating a molecular detection apparatus according to a first embodiment. The molecular detection apparatus 1 shown in FIG. 1 is an apparatus that detects to-be-detected molecules 2 from a detection target gas 3 containing, for example, to-be-detected molecules (to-be-detected substances) generated from a gas generation source. The molecular detection apparatus 1 includes a collection unit 10, a substitution unit 20, a detector 30 and a discriminator 40. The detection target gas 3 containing to-be-detected molecules 2 is, first, collected by the collection unit 10 of the molecular detection apparatus 1. The collection unit 10 has a collection port for the detection target gas 3 and is connected to the substitution unit 20 through a gas flow channel 11. The collection unit 10 may include a filer for eliminating an impurity such as fine particles or the like contained in the detection target gas 3.

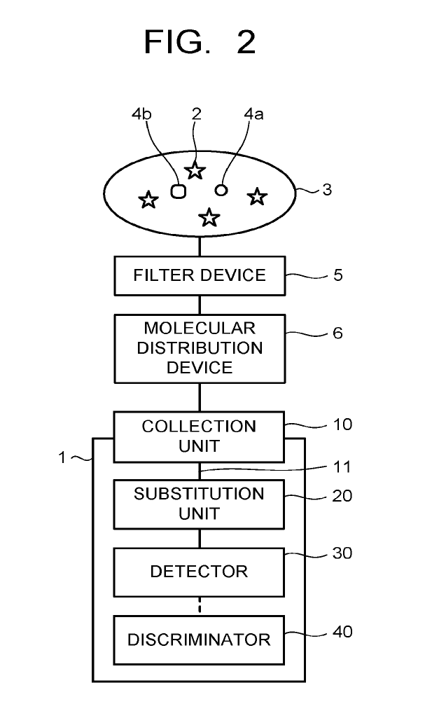

In some cases, the detection target gas 3 contains, as an impurity, a substance that has a molecular weight or a molecular structure or the like similar to the to-be-detected molecule 2. Also, in many cases, the to-be-detected molecule 2 drifting in the air exists, as shown in FIG. 2, in a state that the to-be-detected molecule 2 is immixed with various contaminants 4 (4a and 4b) such as an odorous component or a fine particle or the like. From those perspectives, the detection target gas 3 may be sent to the molecular detection apparatus 1 after the detection target gas 3 is preprocessed by a filer device 5 or a molecular distribution device 6 or the like in advance.

For the filter device 5 out of the preprocessor devices, generally-used moderate high performance filter or the like is used. The filter device 5 eliminates a particulate substance such as a fine particle or the like contained in the detection target gas 3. The detection target gas 3, from which the particular substance is eliminated in the filter device 5, is then sent to the molecular distribution device 6. For the molecular distribution device 6, an apparatus can be used that ionizes the detection target gas 3 to allow the detection target gas 3 to form an ionized substance group, applies voltage to the ionized substance group to allow the ionized substance group to fly at a speed proportional to the mass thereof, and separates an ionized substance of the to-be-detected molecule 2 from the ionized substance group using a flight speed based on the mass difference among ionized substances and a time-of-flight thereof. For this kind of molecular distribution device 6, a device including an ionization unit, a voltage applying unit, and a time-of-flight separation unit may be used.

The detection target gas 3 containing the to-be-detected molecules 2 is collected by the collection unit 10 directly, or alternatively after the detection target gas 3 is preprocessed by a device such as the filter device 5 or the molecular distribution device 6 or the like. The to-be-detected molecules 2 collected by the collection unit 10 are then sent to the substitution unit 20 thorough the gas flow channel 11. The substitution unit 20 substitutes a part of a molecular structure of at least a part of the to-be-detected molecules 2 to generate a substitution product. The substitution unit 20 substitutes, as will be described below in detail, the to-be-detected molecule 2 that is hard to be captured by the organic probe. In some cases, a part of the to-be-detected molecules 2 passing through the substitution unit 20 are not substituted by the substituting unit 20. A particular configuration or a function of the substitution unit 20 and further the substitution state or the like of the to-be-detected molecules 2 will be described below in detail. The substitution product of the to-be-detected molecules 2 generated in the substitution unit 20 and the to-be-detected molecules 2 that has not been substituted in the substitution unit 20 are then introduced into the detector 30.

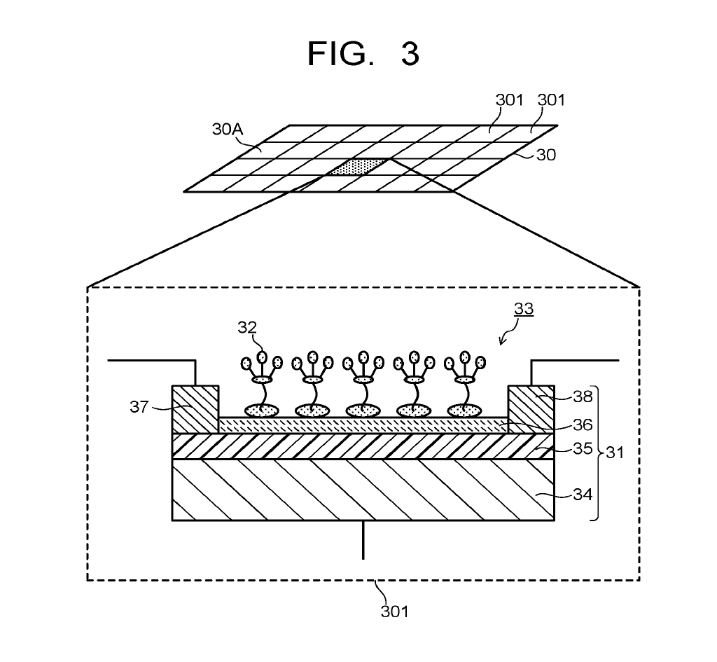

The detector 30 includes, as shown in FIG. 3, a detection surface 30A which is partitioned into a plurality of detection cells 301. The detection surface 30A of the detector 30 is arranged towards an output port (not shown) for the substitution product of the substitution unit 20. Each of the plurality of detection cells 301 includes a detection element 33 having a sensor unit 31 and an organic probe 32 disposed at the sensor unit 31. FIG. 3 illustrates the detection element 33 in which the graphene field effect transistor (GFET) is used for the sensor unit 31.

The GFET serving as the sensor unit 31 includes a semiconductor substrate 34 which functions as a gate electrode, an insulating film 35 provided as a gate insulating layer on the semiconductor substrate 34, a graphene layer 36 provided as a channel on the insulating film 35, a source electrode 37 provided at one end of the graphene layer 36, and a drain electrode 38 provided at the other end of the graphene layer 36. The organic probe 32 is provided on the graphene layer 36 of the GFET 31. The to-be-detected molecule 2 or the substitution product guided into the detector 30 is captured by the organic probe 32 on the graphene layer 36. With electrons being moved from the to-be-detected molecule 2 or the substitution product captured by the organic probe 32 to the GFET 31, the electric detection is carried out. In this way, an intended to-be-detected molecule 2 is detected in a selective manner.

An organic substance constituting the organic probe 32 has a dissolvable property in solvent. Thus, it is possible to arrange the organic probe 32 on the graphene layer 36 by applying on the graphene layer 36 solution in which the organic substance is dissolved. In order to facilitate to achieve an interaction with the graphene, the organic probe 32 has preferably a portion having a certain structure such as a pyrene ring. The molecule having the structure such as the pyrene ring interacts with a hexagonally shaped π electron system constituted with carbon of the graphene to form an interaction state of so-called π-π stacking. The π-π stacking is formed between the pyrene ring and the graphene by dissolving a probe molecule with a low concentration in the solvent and applying the solvent on the graphene, and the probe molecules are aligned and fixed on the graphene 36. By use of this kind of the self-alignment action, it is possible to arrange the organic probe 32 on the graphene layer 36.

When the to-be-detected molecule 2 or the substitution product is captured by the organic probe 32 provided on the graphene layer 36, an output from the GFET 31 changes. When the graphene has a one layer, as it means the zero-gap, normally between the source electrode 37 and the drain electrode 38 continues to be electrified. On the other hand, when the number of graphene layers increases to two or three layers, although the bang gap is generated, such band gap in an actual system is relatively smaller than those considered from the strict theoretical value. When the gate insulating layer 35 has the dielectric constant approximately similar to the silicon dioxide film, in many cases between the source electrode 37 and the drain electrode 38 continues to be electrified. The graphene layer 36 is not limited to the single layer structure of the graphene, but alternatively may be constituted with a laminated body having approximately equal to or less than five layers.

The to-be-detected molecule 2 or the substitution product flying in the vicinity of the organic probe 32 is attracted to the organic probe 32 by the force of hydrogen bond or the like, and in some cases, contacts the organic probe 32. When the contact of the to-be-detected molecule 2 or the substitution product occurs, then an interchange of electrons occurs with the organic probe 32, and an electrical change is transmitted to the graphene layer 36 contacting the organic probe 32. The electrical change transmitted from the organic probe 32 to the graphene layer 36 disturbs the flow of electricity between the source electrode 37 and the drain electrode 38 so that the GFET 31 functions as a sensor.

With the GFET 31 using the graphene layer 36 as a channel being employed, even an extremely slight electrical change appears significantly as an output. As a result, it is possible to constitute the detection element 33 with higher sensitivity. The sensor using the GFET 31 also has a tendency to electrify between the source electrode 37 and the drain electrode 38 without applying voltage to the gate electrode 34, because the graphene has a property as the zero-gap semiconductor. Thus, such sensor can function as it is. Nevertheless, normally between the source electrode 37 and the drain electrode 38 is electrified in a state that the voltage is applied to the gate electrode 34, and the electrical change of the gate electrode 34 is observed when the organic probe 32 has captured the to-be-detected molecule 2 or the substitution product.

In the above mentioned detection of the to-be-detected molecule 2 by the detection element 33, as the travelling of the electron is higher to the GFET 31 from the to-be-detected molecule 2 or the substitution product captured by the organic probe 32, the function as the sensor becomes higher. The sensor using the GFET 31 is considered to be the FET sensor with the highest sensitivity, and is capable of improving the sensitivity approximately three times compared to a sensor using the carbon nanotube. As a result, it is possible to detect the to-be-detected molecule 2 with higher sensitivity by using the detection element 33 that combines the GFET 31 with the organic probe 32.

FIG. 3 illustrates a detection surface 30A on which a plurality of detection cells 301 are arranged in a grid shape (array shape). It however does not mean to limit the present embodiment. A plurality of detection cells 301 may be linearly arranged. Among the organic probes 32 respectively provided on the graphene layers 36 of the plurality of detection units 301, at least a part of the organic probes 32 have a different binding strength with the to-be-detected molecule 2 or the substitution product one another. In other words, the plurality of detection cells 301 include a plurality of organic probes 32 that have different binding strength with the to-be-detected molecule 2 or the substitution product one another. All of the organic probes 32 may have different binding strengths with the to-be-detected molecule 2 or the substitution product one another. Alternatively, a part of the organic probes 32 may have a different binding strength with the to-be-detected molecule 2 or the substitution product one another. Yet alternatively, in place of the organic probes 32 that have different binging strengths with the to-be-detected molecule 2 or the substitution product one another, the density of the organic probes 32 may be changed on the graphene layers 36 one another.

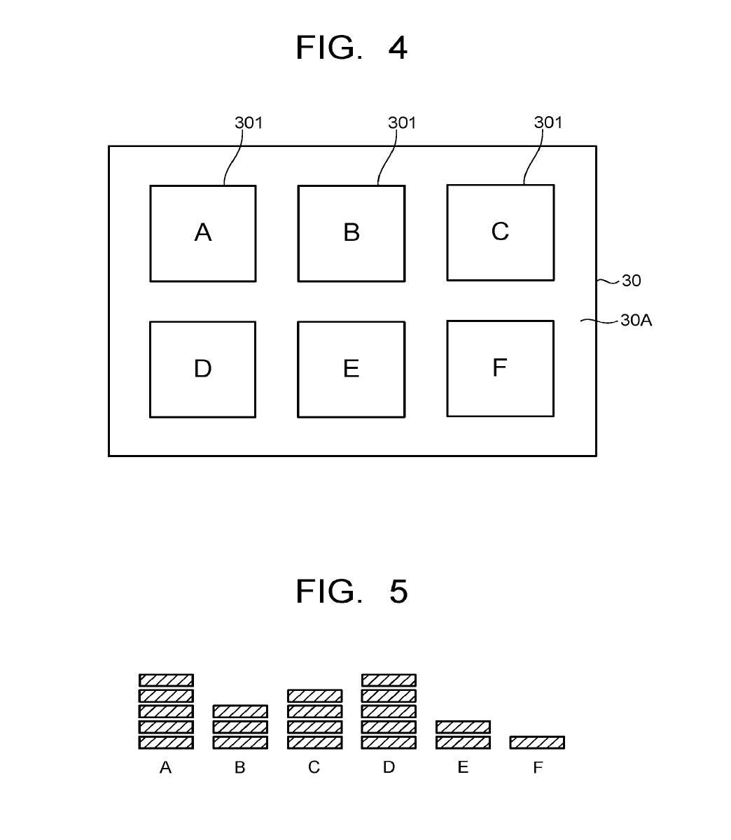

FIG. 4 illustrates a sensor having a grid shape in which the detection surface 30A of the detector 30 is partitioned into six detection cells 301, that is, a detection cell A, a detection cell B, a detection cell C, a detection cell D, a detection cell E, and a detection cell F. Out of the detection cells A to F, the different types of organic probes 32, in other words, a plurality of organic probes 32 having the different binding strengths with the to-be-detected molecule 2 or the substitution product one another, are provided for at least a part of detection cells. The plurality of organic probes 32 interact with the to-be-detected molecule 2 or the substitution product, respectively, and have different signal intensity of the detection signals one another, respectively, as they have different acting strength (binding strength) with the to-be-detected molecule 2 or the substitution product. FIG. 5 illustrates an example of the detection signals detected by the detection cells A to F, respectively. The detection signals from the detection cells A to F have different signal intensities one another based on the binding strengths of the organic probes 32 with the to-be-detected molecule 2 or the substitution product.

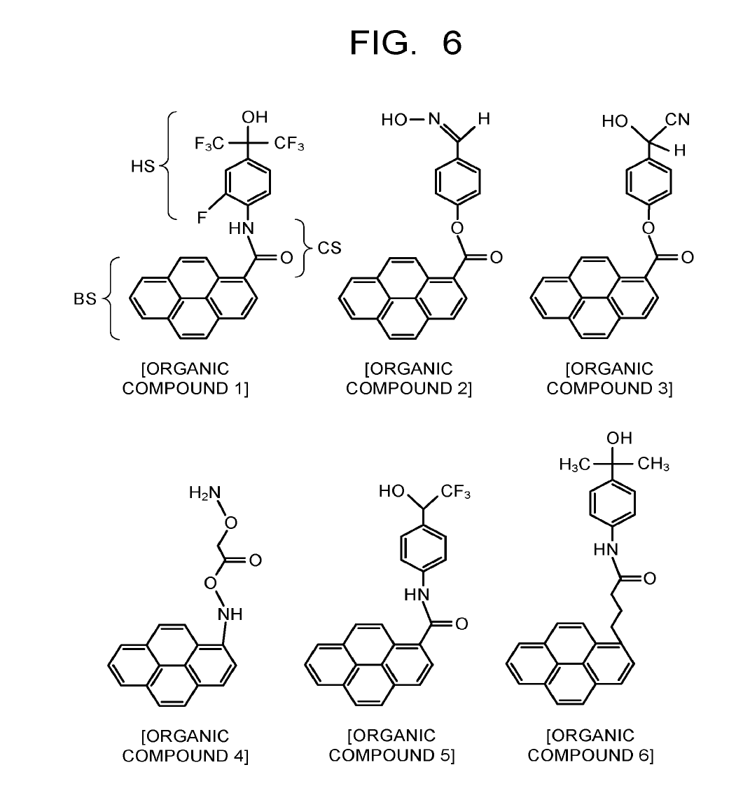

FIG. 6 illustrates an example of the organic probes 32 provided on the graphene layers 36 of the detection cells A to F, respectively. Out of the organic compounds constituting the organic probes 32, the organic compound 1 to 3, 5 and 6 has a hydroxy group (—OH) as a reactive group with respect to the to-be-detected molecule 2 or the substitution product. The organic compound 4 has an amino group (—NH₂) as a reactive group. However, it should be noted that, when this kind of reactive group is solely used, the reactive group hardly reacts with the gas component. In order to enhance the hydrogen bonding property, an organic compound in which a functional group (neighboring group) having an excellent inductive effect is introduced into a neighboring portion of the reactive group is used.

As the neighboring group to the hydroxy group (—OH) as the reactive group, an alkyl group substituted with a fluorine atom such as a trifluoromethyl group (—CF₃) or a hexafluoroethyl group (—C₂F₅) or the like, a functional group containing nitrogen such as a cyano group (—CN), a nitro group (—NO₂), or —CHN group or the like, or an alkyl group such as a methyl group (—CH₃) or an ethyl group (—C₂H₅) or the like may be used. The organic compound 1 and 5 have the trifluoromethyl groups (—CF₃) as the neighboring groups to the reactive groups (—OH), respectively. The organic compound 2 has a —CHN— OH group as the functional group containing the reactive group. The organic compound 3 has the cyano group (—CN) as the neighboring group to the reactive group (—OH). The organic compound 6 has the methyl group (CH₃) as the neighboring group to the reactive group (—OH). As the neighboring group to the amino group (—NH₂) as the reactive group, an ether linking group (—O—) may be used. The organic compound 4 has a —O—NH₂ group as the functional group containing the reactive group.

The organic compounds 1 to 6 shown in FIG. 6 are examples of an organic compounds constituting the organic probe 32, and the organic probe 32 is not limited to the organic compounds 1 to 6 shown. The organic probe 32 is preferably constituted with, as shown in the organic compound 1 in FIG. 6, an organic compound having a head portion HS, which has a reactive group such as the hydroxy group or the amino group or the like and the above mentioned neighboring group, a base portion BS, which serves as an installation portion for the graphene layer 36 or the like, and a connecting portion CS, which connects the head portion HS to the base portion BS. The head portion HS is preferably a monovalent aromatic hydrocarbon group having the reactive group and the neighboring group, and more preferably a phenyl group having an alkyl group in which the reactive group and the neighboring group are bound to the same carbon (carbon number: approximately 1 to 5).

The base portion BS is preferably a monovalent substituted or unsubstituted polycyclic aromatic hydrocarbon group having a polycyclic structure such as a pyrene ring, an anthracene ring, a naphthacene ring, or a phenanthrene ring or the like, and more preferably a substituted or unsubstituted pyrene group. The connecting portion CS may be a bivalent group. The connecting portion CS may be an alkylene group such as a methylene group or an ethylene group or the like. The connecting portion CS has preferably an ether bond (—O—), an ester bond (—C(═O)O—), a carbonyl bond (—CO—), an amide bond (—NH—CO—), an imide bond (—CO—NH—CO—) or the like, and more preferably has the amide bond.

In the organic compound constituting the above mentioned organic probe 32, the binding strength with the to-be-detected gas molecule 2 or the substitution product can be regulated depending on the type of reactive group, the type or the number of the neighboring group to the reactive group. For example, the organic compound 6 has a different neighboring group (CH₃ group) from a neighboring group (CF₃ group) of the organic compound 1. The trifluoromethyl group achieves an effect to enhance an activity of the reactive group (OH group) with fluorine having a higher electronegative degree, while the methyl group has less such effect. In light of the above observation, it is possible to obtain the different binding strengths with the to-be-detected molecule 2 one another. Also, as the number of neighboring group (CF₃ group) of the organic compound 5 is different from the number of the neighboring group of the organic compound 1, the binding strengths with the to-be-detected molecule 2 differ each other. Yet furthermore, as the type of functional group containing the reactive group among organic compounds 2 to 4 differ from the type of functional group of the organic compound 1, the binding strengths with the to-be-detected molecule 2 differ one another.

As described above, the binding strength with the to-be-detected molecule 2 or the substitution product can be regulated depending on the type of the organic compound constituting the organic probe 32. It is possible to regulate the binding strength with the to-be-detected molecule 2 or the substitution product by regulating the density of the organic probes 32 provided on the detection cell 301. The signal intensities of the detection signals from the detection cells A to F differ one another based on the difference in the binding strengths with the to-be-detected molecule 2 or the substitution product of the organic probes 32.

The signals respectively detected by the detection cells A to F are sent to a discriminator 40 and undergoes the signal processing. The discriminator 40 transforms the detection signals from the detection cells A to F into intensities, and then analyzes a signal pattern based on the difference in the intensities of those detection signals (for example, pattern of six detection signals shown in FIG. 5). The discriminator 40 stores a signal pattern corresponding to a to-be-detected substance. Thus, the discriminator 40 discriminates the to-be-detected molecule 2 detected by the detector 30 by comparing the stored signal pattern and a signal pattern detected by the detection cells A to F. This type of signal processing method is referred to as a pattern recognition method. According to the pattern recognition method, it is possible to detect and discriminate the to-be-detected molecule 2 with the signal pattern specific to the to-be-detected substance as, for example, a fingerprint inspection. As a result, it is possible to detect a gas component with the extremely low concentration in the order of ppt to ppb (to-be-detected molecule 2) in a selective manner with higher sensitivity.

By applying the above mentioned pattern recognition method, even in the case that an impurity is immixed into the detection target gas to be introduced into the detector 30, still it is possible to detect and discriminate the to-be-detected molecule 2 in a selective manner with higher sensitivity. For example, in the case that the to-be-detected molecule 2 is dimethyl methylphosphonic acid (DMMP, the molecule weight: 124), which is a typical material for a noxious organic phosphorous compound, there are an agricultural chemical containing phosphoric acid such as dichlorvos having a similar chemical structure and an organic phosphorous pesticide with a lot of usage examples such as malathion, chlorpyrifos, or diazinon or the like. In order to prevent an erroneous detection of those substances, it is effective to discriminate with the signal patterns as shown in FIG. 5. In other words, because the signal patterns detected by the detection cells A to F differ one another depending on the above mentioned respective substances, it is possible to detect the detection target substance in a selective manner with higher sensitivity by applying the pattern recognition method even when an impurity is immixed that has a close molecular weight and a similar constituent element.

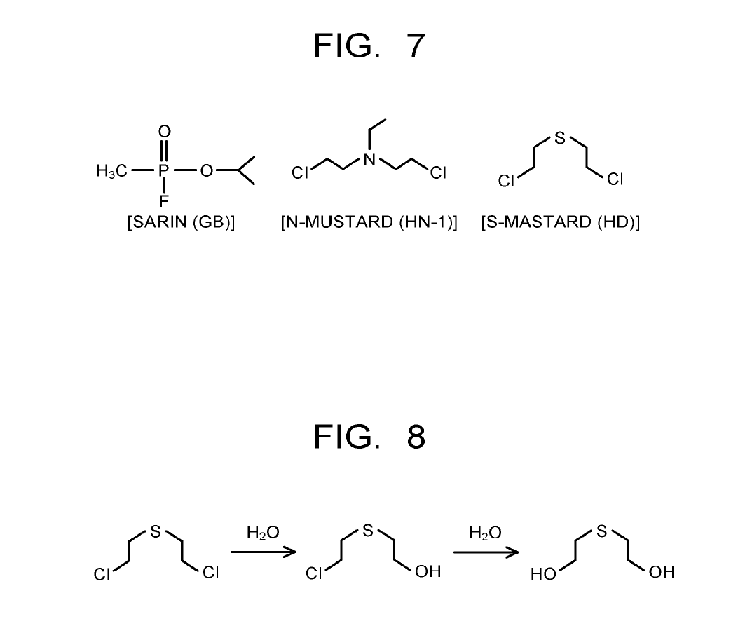

In capturing the to-be-detected molecules 2 by the above mentioned organic probe 32, the to-be-detected molecules 2 fall into those easy to be captured and those hard to be captured depending on the type thereof. This difference depends on an intramolecular structure of the gas molecules, and primarily relates to the polarity of the whole molecule created between neighboring atoms in the molecule. FIG. 7 illustrates an example of gas molecule detected by the molecular detection apparatus 1 according to the present embodiment. For example, an organic phosphorous compound contained in sarin (GB) or the noxious agricultural chemical has a structure containing a double bond of phosphorus and oxygen (P═O) or a single bond of phosphorus and oxygen (P—O). This type of bond structure of phosphorus and oxygen has relatively large polarity so that it is more likely to obtain an interaction with the organic probe 32 by the hydrogen bond. For this reason, the bond structure of phosphorus and oxygen is a molecule that is easy to be captured by the organic probe 32. Also, nitrogen type mustard (HN−1), which serves as the noxious gas, has a structure containing a single bond of nitrogen and carbon (N—C). This type of bond structure has also relatively large polarity so that it is more likely to obtain an interaction with the organic probe 32 by the hydrogen bond.

On the other hand, sulfur type mustard (HD) has a single bond of sulfur and carbon (S—C bond), which has relatively small polarity (electrical deviation). Moreover, a molecular structure of the sulfur type mustard (HD) has a good left-right symmetry so that it also allows the smaller polarity from this viewpoint. This kind of gas molecule (to-be-detected molecule 2) is hard to obtain the interaction with the organic probe 32 so that such gas molecule is a molecule that is hard to be captured by the organic probe 32. As described above, the to-be-detected molecules 2 have different property one another depending on the molecular structures thereof. In this regard, the substitution unit 20 changes the molecular chemical structure of the to-be-detected molecule 2 that is hard to be captured by the organic probe 32 and remakes it into a structure that is relatively easy to be captured. As a result, it make it easier to capture and detect thereupon by the organic probe 32.

In the molecular detection apparatus 1 according to the embodiment, the substitution unit 20 generates the substitution product having a molecular structure that is easy to be captured by the organic probe 32 by changing a part of a molecular structure of the to-be-detected molecule 2 that is hard to be captured as it is by the organic probe 32. For example, in the case of the sulfur type mustard (HD), as shown in FIG. 8, at least one of chlorine groups (—Cl) as terminal groups is substituted with a hydroxy group (—OH) so as to be transformed into a molecule having a strong interaction with the organic probe 32. As the hydroxy group has a large polarity as it is, the transformed molecule becomes easier to be captured by the organic probe 32 with the hydrogen bond.

As described above, the substitution unit 20 substitutes a part of molecular structure of the to-be-detected molecule 2 that is hard to be captured by the organic probe 32 to generate a new molecule, in other words, performs the molecular transformation. Thus, it makes it possible to generate the substitution product having a molecular structure that is easy to be captured by the organic probe 32. As the to-be-detected molecule 2 that is hard to be captured by the organic probe 32, an organic compound having a portion in which sulfur and carbon bond together (S—C bond portion), of which polarity is small, and a portion of a chlorine group (—Cl) as the terminal group can be used. However, it is not limited to such organic compound. For detecting the to-be-detected molecule 2 for the substitution product, the discriminator 40 stores a signal pattern corresponding to the substitution product and compares the stored signal pattern to a signal pattern based on an intensity difference of detection signals from a plurality of detection cells A to F when the substitution product as the to-be-detected molecule 2 is captured by the organic probe 32. Accordingly, it makes it possible to detect the to-be-detected molecule 2 in a selective manner with higher sensitivity.

For the substitution unit 20 for substituting a part of the molecular structure of the to-be-detected molecule 2, for example, a container such as a column or the like is applied that is filled up with porous substances of a metal organic composition body (metal organic framework: MOF), which is a complex of the metal and the organic substance, or fine particles of vanadium oxide. As a concrete example of the MOF, a composition body that contains copper (Cu ion) as the metal and an organic compound (organic ligand) having a carbonyl group as the organic substance can be used. It is possible to substitute the chlorine group of the to-be-detected molecule 2 with the hydroxy group by allowing the detection target gas 3 containing the to-be-detected molecule 2 to pass through inside the column filled up with those kinds of MOF or fine particles of vanadium oxide. Because the MOF has a porous property as it is, it is possible to substitute the chlorine group with the hydroxy group by allowing the detection target gas 3 to pass through inside vacancies of the MOF. As for the fine particles of vanadium oxide, it is possible to substitute the chlorine group with the hydroxy group by allowing the detection target gas 3 to pass through between the fine particles of vanadium oxide filled up inside the column.

Water involves the above mentioned substitution reaction from the chlorine group to the hydroxy group, and the reaction is accelerated when water is involved. For this reason, the substitution unit 20 is preferably provided with a humidifying mechanism configured to regulate an amount of moisture and a dew meter configured to monitor the amount of moisture. The humidifying mechanism increases the amount of moisture equal to or greater than several % inside the column filled up with the MOF or the fine particles of vanadium oxide to maintain the transformation efficiency (substitution efficiency) of the to-be-detected molecule 2 by the substitution unit 20. Accordingly, it makes it possible to use the molecular detection apparatus 1 even in a dried region to the utmost, for example, a desert region. Also, with the humidifying mechanism and the dew meter being used, it is possible to suppress the variation in the transformation efficiency due to the change in the amount of moisture under a normal environment. As a result, it makes it possible to achieve a satisfactory transformation efficiency of the to-be-detected molecule 2 in a stable manner.

It should be noted that the to-be-detected molecule 2 that is easy to be captured by the organic probe 32 derived from the original molecular structure can be detected by the detector 30 without causing problems even if it is sent to the detector 30 through the substitution unit 20. Accordingly, the molecular detection apparatus 1 is capable of detecting both of the to-be-detected molecule 2 that is hard to be captured by the organic probe 32 and the to-be-detected molecule 2 that is easy to be captured by the organic probe 32. However, in the case that the substitution unit 20 is applied, it is likely to generate the loss of pressure to reduce an amount of passing gas when the detection target gas 3 passes through inside the container filled up with the MOF or the fine particles of vanadium oxide. For this reason, in order to improve the detection accuracy or to reduce the detection time of the to-be-detected molecule 2 that is easy to be captured by the organic probe 32, as will described below in a second embodiment, it is preferable to provide dual gas flow channels, namely, a gas flow channel in which the substitution unit 20 is arranged and a gas flow channel that directly connects the collection unit 10 to the detector 30.

In the molecular detection apparatus 1 according to the first embodiment, it is possible to detect the gas molecule with the extremely low concentration in the order of ppt to ppb in a selective manner with higher sensitivity by applying the pattern recognition method. In addition, it is possible to detect the gas molecule with higher sensitivity irrespective of the type of the gas component as the to-be-detected molecule 2 by substituting, with the substitution unit 20, a part of the molecular structure of the to-be-detected molecule 2 that is hard to be captured by the organic probe 32 and transforming into the substitution product that is easy to be captured by the organic probe 32. Also, it is possible to downsize the molecular detection apparatus 1 by improving the detection sensitivity and the detection accuracy at the detector 30 and the discriminator 40. As a result, it is possible to provide the molecular detection apparatus 1 that satisfies both of the portability and the detection accuracy. This kind of molecular detection apparatus 1 effectively fulfills its function at various field sites such as a disaster site or a site of an act of terrorism or the like.

### Second Embodiment

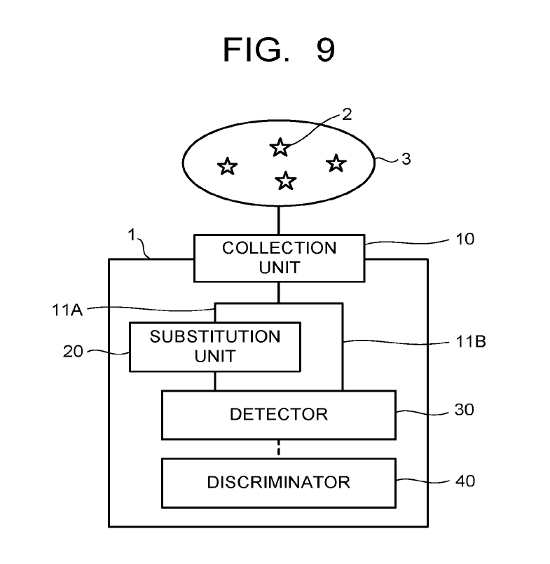

FIG. 9 is a block diagram illustrating a molecular detection apparatus according to a second embodiment. The molecular detection apparatus 1 illustrated in FIG. 9 comprises, similarly to the first embodiment, a collection unit 10, a substitution unit 20, a detector 30 and a discriminator 40. The configurations of those units 10, 20, 30 and 40 have been already described in detail in the first embodiment. Gas flow channels from the collection unit 10 to the detector 30 employ dual system having a first gas flow channel 11A and a second gas flow channel 11B. The first gas flow channel 11A is provided with the substitution unit 20, and connected from the collection unit 10 through the substitution unit 20 to the detector 30. The second gas flow channel 11B is directly connected from the collection unit 10 to the detector 30.

With respect to a to-be-detected molecule (first to-be-detected molecule) 2 that is hard to be captured by the organic probe 32, similarly to the first embodiment, a part of the molecular structure of the first to-be-detected molecule 2 is substituted by the substitution unit 20 through the first gas flow channel 11A, and the substitution product is generated and sent to the detector 30. On the other hand, with respect to a to-be-detected molecule (second to-be-detected molecule) 2 that is easy to be captured by the organic probe 32, the second to-be-detected molecule 2 is directly sent to the detector 30 through the second gas flow channel 11B. When the detection target gas 3 contains both the first and second to-be-detected molecules, both of them may be sent to the detector 30 at the same time. Alternatively, the gas flow channels 11A and 11B may be provided with a valve or the like for switching, and the to-be-detected molecule 2 or the substitution product may be sent to the detector 30 only through one of the gas flow channels 11A, 11B depending on the detection target substance.

When the substitution product, which is substituted from the first to-be-detected molecule, and the second to-be-detected molecule are sent to the detector 30 at the same time, although the detection signal patterns are obtained in a superimposed state, it is possible to detect the substitution product, which is substituted from the first to-be-detected molecule, and the second to-be-detected molecule from such detection signal patterns. Also, in order to perform the detection with further higher accuracy, it is preferable to allow only one of the gas flow channels to be set to an open state depending on the detection target substance. With this configuration, the second to-be-detected molecule, which does not require to substitute a part of the molecular structure thereof, is sent to the detector 30 without passing through the substitution unit 20, which is likely to entail the reduction or the like of the passing gas amount. As a result, it makes it possible to improve the detection accuracy and to reduce the detection time. In other words, it makes it possible to detect both of the to-be-detected molecule that is hard to be captured by the organic probe 32 (first to-be-detected molecule) and the to-be-detected molecule that is easy to be captured by the organic probe 32 (second to-be-detected molecule) in a selective manner with higher sensitivity.

In the following description, specific examples and evaluation results thereof will be described.

### Example 1

As a material used for the substitution unit, MOF is synthesized as will be described below. First, trimesic acid of 5 g and copper nitrate of 10 g are dissolved in solvent of 250 mL into which dimethyl formamide (DMF), ethanol and water are immixed. The solution is put into a round bottom flask and warmed in a warm bathing to react at 85 degrees Celsius for 20 hours. When a solid constituent is settled, the temperature is lowered and the solution is left still for some moments. After then, the solvent component, as supernatant solution, is decanted. The remaining solid constituent is cleaned by DMF for several times, immersed in dichloromethane and then left at room temperature for several days. The solid substance is filtered and recovered. The recovered solid substance is vacuum dried using the dry ice trap. The solid substance is heated to 170 degrees Celsius to remove the residual component of the solvent. In this way, 10 g of MOF of a blue solid substance (HKUST-1: copper benzene-1,3,5-tricarboxylate) is obtained. After the MOF is crushed, a container having a cylindrical shape is filled up with the crushed MOF to constitute a substitution unit.

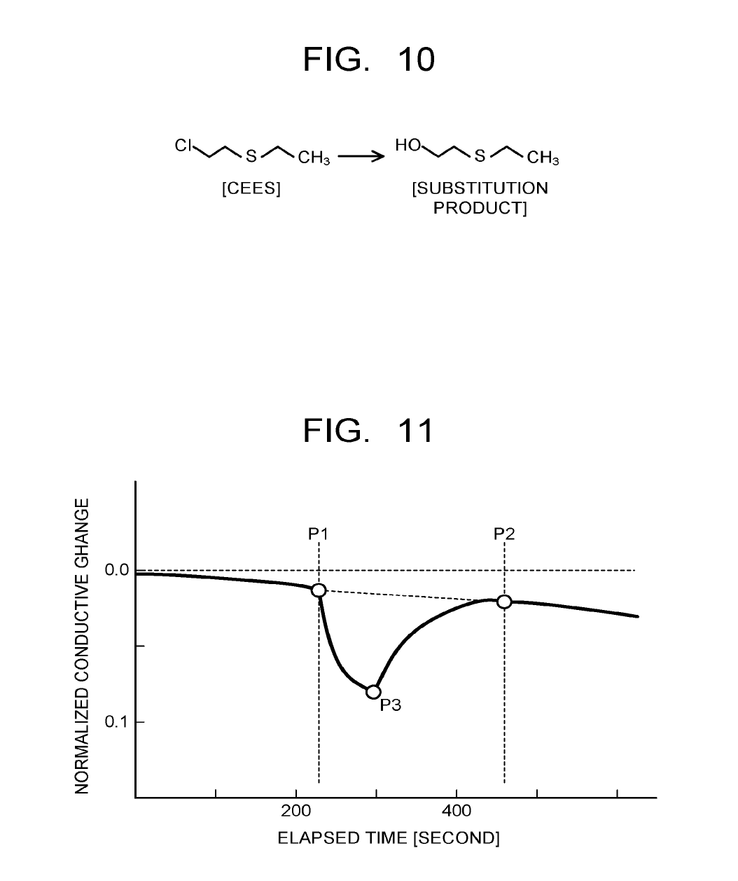

In Example 1, 2-chloroethyl ethylsulfide (CEES) is prepared, of which molecular structure is shown in FIG. 10, as an alternative substance to the sulfur type mustard (HD) as the to-be-detected molecule. The CEES is attenuated with the nitrogen gas such that the CEES has the concentration of 1 ppm, and then immixed with moisture vapor after passing through the molecular distribution device. This kind of gas component is caused to pass through inside the container filled up with the above mentioned MOF. Although the passage of the gas component may be once, it is more effective if it is provided with a structure configured to circulate the gas component several times. With the gas component containing the CEES passing through inside the container filled up with the MOF, as shown in FIG. 10, the chlorine group at a terminal is substituted with the hydroxy group. The gas component containing the substitution product in which the chlorine group of the CEES is substituted with the hydroxy group is sent to a detector having a detection element in which a GFET and an organic probe is combined, as will be described below.

A detection element in which the GFET and the organic probe are combined is prepared as described below. The graphene layer is formed by a transcription onto the substrate by peeling off method from the graphite. Alternatively, the graphene layer is formed by depositing onto a surface of metal by use of the chemical vapor deposition (CVD) method. The graphene with single layer or multiple layers deposited onto the metal surface is transcribed onto a polymer film, and then transcribed again onto a semiconductor substrate for preparing a desired field effect transistor (FET). For example, the graphene is formed by the CVD that flows methane gas onto a surface of a copper foil under the condition of approximately 1000 degrees Celsius.

Subsequently, polymethyl methacrylate film is applied at 4000 rpm by the spin coat method, and the copper foil at an opposite face is etched with ammonium persulfate solution of 0.1 M so that a graphene film floating in the solution is recovered. By doing this, the graphene film is transcribed onto a side of polymethyl methacrylate film. After a surface of the graphene is sufficiently cleaned, it is again transcribed onto a silicon substrate. A superfluous polymethyl methacrylate film is removed by dissolving with acetone. A resist is applied onto the graphene transcribed onto the silicon substrate to perform the patterning, and an electrode pattern with a distance between electrodes of 10 μm is formed by an oxygen plasma. The FET structure having the source electrode and the drain electrode is formed by vapor-depositing the electrodes. In this way, the graphene is arranged onto the oxidized film formed on the surface of the silicon substrate so that a sensor structure having the FET in which the graphene is sandwiched between the source electrode and the drain electrode, and the gate electrode is provided at the silicon substrate.

The graphene sensor also has a tendency to electrify between the source and the drain without applying voltage to the gate electrode, because the graphene has a property as the zero-gap semiconductor. For this reason, the graphene functions as a sensor and is capable of obtain a detection signal with a substance colliding against the graphene. Nevertheless, normally between the source and the drain is electrified in a state that the voltage is applied to the gate electrode, and the electrical change of the gate electrode is observed when the substance contacts.

Subsequently, the organic probe is provided on the surface of the graphene. More particularly, the organic probe is provided by dissolving with the concentration of 10 nM in methanol solution and immersing the graphene sensor face therein for several minutes. Organic compounds 1 to 6 shown in FIG. 6 are used for the organic probe. In the Example 1, as shown in FIG. 4, six detection cells A to F are provided on the detection surface of the detector, and different organic compounds 1 to 6 shown in FIG. 6 are provided for the respective cells as the organic probes. As described above, those organic compounds have different binding strengths with the to-be-detected molecule (CEES) one another.

The gas component containing the substitution product of the to-be-detected molecule (CEES) is introduced into the detector having the above mentioned detection cells A to F to detect the CEES. The substitution products of the to-be-detected molecule (CEES) are captured by the organic probes of the detection cells A to F, respectively. The organic probes of the detection cells A to F have different binding strengths with the substitution products of the to-be-detected molecule (CEES) one another, so that signals detected at the gate electrodes differ one another, respectively. Detection results by the detection cells A to F are sent to a discriminator for signal processing to transform into the signal intensities. Various methods are conceivable for transforming into an intensity, and here the intensity is set as a value calculated from an area defined by P1, P2 and P3, which is a tip of a peak, in FIG. 11. Nevertheless, it is not limited to this method.

As shown in FIG. 5, the recognition results are output in relative intensity displays. FIG. 5 illustrates the results in which the substitution product of the CEES was measured as the to-be-detected substance. In the pattern recognition, different intensities from respective cells are collectively analyzed, and a signal intensity pattern specific to each of the to-be-detected molecule is obtained. It is possible to detect the to-be-detected substance (gas molecule) with extremely low concentration in the order of ppt to ppb in a selective manner with higher sensitivity, by discriminating the to-be-detected substance based on the signal pattern according to this kind of signal intensity difference. In addition, as the substitution unit is used, it is possible to also detect the gas molecule that is hard to be captured by the organic probe with as is molecular structure thereof.

### Example 2

As a material used for the substitution unit, fine particles of vanadium oxide were prepared as will be described below. First, vanadium oxide of 1.5 g and dodecyl amine of 1.8 mL are dissolved in ethanol of 25 mL. The solution has been continuously stirred for approximately 7 hours, and purified water of 70 mL is added. The substance obtained has been heated at 180 degrees Celsius for one week. Then a green solid substance is obtained. The obtained solid substance is cleaned with the purified water and ethanol, and then dried at 50 degrees Celsius in order to eliminate moisture. Nanorod shaped vanadium oxide obtained in this way is crushed to fill up a cylindrical shaped container to form the substitution unit.

Similarly to Example 1 except for the above mentioned substitution unit filled up with the fine particles of vanadium oxide being used, Example 2 performs a process for substituting a part of molecular structure of the CEES to generate the substitution product, a process for detecting the gas component containing such substitution product by the detector, and a process for discriminating by the discriminator. The detector and the organic probe used therefor are similar to those in Example 1. As a result, as shown in FIG. 5, relative signal intensity patterns are obtained as the recognition results. Detection of the CEES is determined from the signal intensity patterns shown in FIG. 5. Similarly to Example 1, it is determined that the CEES, which is hard to be captured with as is molecular structure thereof, can be detected with higher accuracy, by employing the substitution unit using the fine particles of vanadium oxide as well.

While certain embodiments of the present invention have been described, these embodiments have been presented by way of example only, and are not intended to limit the scope of the invention. Indeed, the novel embodiments described herein may be embodied in a variety of other forms. Various omissions, substitutions, changes and modifications may be made to the embodiments described herein without departing from the spirit and scope of the present invention. The appended claims and their equivalents are intended to cover such embodiments and modifications as would fall within the scope and spirit of the invention.

What is claimed is:

1. A molecular detection apparatus, comprising:
a collection unit collecting detection target gas containing molecules to be detected;
a substitution unit substituting a part of a molecular structure of at least a part of the molecules collected by the collection unit to generate a substitution product;
a detector including a plurality of detection cells each having a sensor unit and an organic probe disposed at the sensor unit, the organic probe capturing the molecules or the substitution product sent from the substitution unit; and
a discriminator discriminating the molecules by a signal pattern based on an intensity difference of detection signals generated with the molecules or the substitution product being captured by the organic probes of the plurality of detection cells.

2. The apparatus of claim 1, wherein the sensor unit comprises a field effect transistor including a graphene layer and a source electrode and a drain electrode both connected to the graphene layer, and the organic probe is provided on the graphene layer.

3. The apparatus of claim 1, wherein the plurality of detection cells have a plurality of the organic probes having different binding strengths with the molecule or the substitution product one another.

4. The apparatus of claim 1, wherein
the molecule includes a portion in which sulfur and carbon are bound and a chlorine group as a terminal group,
the substitution unit includes a container filled up with a porous substance of a metal organic framework as a composite body of a metal and an organic substance, and
the chlorine group is substituted into a hydroxy group with the molecule passing through inside the porous substance.

5. The apparatus of claim 4, wherein the metal organic framework contains copper as the metal and an organic compound including a carbonyl group as the organic sub stance.

6. The apparatus of claim 4, wherein the substitution unit is provided with a humidifying mechanism.

7. The apparatus of claim 1, wherein
the molecule includes a portion in which sulfur and carbon are bound and a chlorine group as a terminal group,
the substitution unit includes a container filled up with fine particles of vanadium oxide, and
the chlorine group is substituted into a hydroxy group with the molecule passing through between the fine particles of vanadium oxide.

8. The apparatus of claim 7, wherein the substitution unit is provided with a humidifying mechanism.

9. The apparatus of claim 1, further comprising:
a first and a second gas flow channels from the collection unit towards the detector, and wherein
the first gas flow channel is provided with the substitution unit, and the molecules are sent to the detector through the substitution unit in the first gas flow channel,
the second gas flow channel is directly connected to the detector, and the molecules are directly sent to the detector in the second gas flow channel, and
the organic probe captures at least one of the substitution product sent through the first gas channel and the molecules sent through the second gal flow channel.

10. A method for detecting a molecule, comprising:
collecting detection target gas containing molecules to be detected;
substituting a part of a molecular structure of at least a part of the molecules collected by the collection unit to generate a substitution product;
capturing the molecules or the substitution product by organic probes provided in sensor units of a plurality of detection cells; and
discriminating the molecules by a signal pattern based on an intensity difference of detection signals generated with the molecules or the substitution product being captured by the organic probes of the plurality of detection cells.

11. The method of claim 10, wherein the plurality of detection cells have a plurality of the organic probes having different binding strengths with the molecule or the substitution product one another.

12. The method of claim 10, wherein
the molecule includes a portion in which sulfur and carbon are bound and a chlorine group as a terminal group,
a process for generating the substitution product is performed using a porous substance of a metal organic framework as a composite body of a metal and an organic substance, and
the chlorine group is substituted into a hydroxy group with the molecule passing through inside the porous substance.

13. The method for detecting a molecule of claim 10, wherein
the molecule includes a portion in which sulfur and carbon are bound and a chlorine group as a terminal group,
a process for generating the substitution product is performed using fine particles of vanadium oxide, and
the chlorine group is substituted into a hydroxy group with the molecule passing through between the fine particles of vanadium oxide.
